(19) United States
(12) Patent Application Publication
(10) Pub. No.: US 2026/0263806 A1
(43) Pub. Date: Sep. 10, 2026

(54) IMPLANTABLE SYSTEMS AND DEVICES FOR STIMULATION OF A CAROTID SINUS NERVE AND/OR BARORECEPTORS

(71) Applicant: THE ALFRED E. MANN FOUNDATION FOR SCIENTIFIC RESEARCH, VALENCIA, CA (US)

(72) Inventors: Samuel David Bredeson, Sherman Oaks, CA (US); William Brandt, Castaic, CA (US); Joseph Louis Calderon, Santa Clarita, CA (US); Robert J. Greenberg, Los Angeles, CA (US)

(73) Assignee: THE ALFRED E. MANN FOUNDATION FOR SCIENTIFIC RESEARCH, VALENCIA, CA (US)

(21) Appl. No.: 19/555,702

(22) Filed: Mar. 3, 2026

Related U.S. Application Data
(60) Provisional application No. 63/767,496, filed on Mar. 5, 2025.

(51) Int. Cl. A61N 1/36; A61N 1/05

(52) U.S. Cl. CPC A61N 1/36117; A61N 1/0556; A61N 1/36135

(57) ABSTRACT

Implantable systems and devices for stimulation of a carotid sinus nerve (CSN) and/or baroreceptors are disclosed. In one embodiment, an electrode lead includes a cuff having a cuff body configured for placement on or about a carotid artery. The cuff body includes a plurality of electrodes arranged along a first direction and staggered along a second direction transverse to the first direction such that at least some electrodes overlap. The electrode arrangement accommodates anatomical variability in the position of a carotid sinus nerve (CSN) and/or baroreceptors. The cuff body may include fixation features and may be elastically conformable to accommodate variations in carotid artery diameter. The system further includes an implantable pulse generator configured to deliver electrical stimulation.

### CROSS-REFERENCE TO RELATED APPLICATION

This application claims the benefit of U.S. Provisional Application No. 63/767,496, filed Mar. 5, 2025, and entitled “Implantable Nerve Cuff and Method for Carotid Sinus Nerve Stimulation,” which is incorporated herein by reference.

### FIELD OF THE INVENTION

The present disclosure relates to implantable stimulation systems and devices, and more particularly, to implantable systems and devices configured to deliver electrical stimulation to a carotid sinus nerve (CSN) and/or baroreceptors associated with a carotid artery.

### DESCRIPTION OF RELATED ART

Implantable tissue stimulators, which may include an implantable pulse generator (“IPG”) and an electrode lead, are used to treat a wide variety of medical conditions. Electrode leads may include a lead body, a neural interface such as a nerve cuff, and a plurality of electrically conductive contacts (or “electrodes”) disposed on the nerve cuff, although the exact configuration of the lead will depend upon the medical condition being treated.

Implantable tissue stimulators can be used to treat hypertension by delivering pulses of electrical stimulation to the carotid sinus nerve (“CSN”) and/or to baroreceptors located in the carotid sinus region. The carotid sinus is located at the bifurcation of the common carotid artery and plays a crucial role in blood pressure regulation. It contains baroreceptors that detect changes in blood pressure and relay feedback to the brain via the CSN. The brain, in turn, uses this feedback to modulate heart rate and control the constriction/dilation of blood vessels to maintain blood pressure within a homeostatic range. This feedback loop is commonly referred to as the baroreflex response. Supplemental electrical stimulation of the CSN or the baroreceptors can elicit a baroreflex response to reduce high blood pressure.

However, direct stimulation of the CSN poses several challenges. With a diameter of about 0.5 to 1.5 mm, the CSN is a delicate structure that is difficult to stimulate with conventional nerve cuffs, which are usually designed for larger nerves. The CSN is closely associated with the carotid artery and carotid sinus region through fibrous connective tissue, and its precise location and course can vary among patients. These factors obscure surgical access and complicate electrode placement.

Stimulating the CSN is conventionally accomplished using either cuff-style leads that wrap circumferentially around the internal carotid artery (ICA) or relatively smaller neural interfaces, such as C-FINE electrodes, that wrap circumferentially around the CSN itself. Many cuff-style leads are overly constrictive and fail to accommodate changes in vessel diameter during arterial pulsation. Over time, this causes irritation at the implant site and the formation of scar tissue. Due to differences in patient anatomy, cuff-style leads can also be difficult to position to stimulate the CSN effectively. On the other hand, attaching a relatively smaller electrode cuff directly around the CSN requires surgically separating the CSN from the ICA, a technically challenging procedure that carries greater risks.

### SUMMARY

The following summary provides non-limiting, non-exhaustive examples of systems, methods, elements, advantages, and features (collectively, “aspects”) in accordance with the present disclosure. Any or all aspects could be interchangeably applied between examples, and no single aspect is essential to achieve the technical effects/solutions described. Certain aspects are described in the context of a stimulation system configured for stimulating a carotid sinus nerve (CSN) and/or baroreceptors located in a carotid artery or carotid sinus region. However, this disclosure is not limited to methods of treatment, and some aspects could apply to the treatment of other medical conditions or to the modulation of autonomic activity associated with the carotid sinus region or other physiological targets influenced by carotid sinus stimulation.

The present inventors have determined that there is a need for improvements to electrode leads and associated systems for stimulating the CSN and/or baroreceptors located in a carotid artery. They have determined that conventional cuff-style electrode leads do not suitably accommodate interpatient differences in patient anatomy, such as variations in the location and course of the CSN and variability in carotid sinus anatomy, which causes difficulties with cuff placement and stimulation reliability.

The present disclosure provides a stimulation system including an electrode lead with a cuff design that is easy to implant and that better accommodates arterial movement and avoids constriction, and an electrode arrangement that accommodates variable positioning of the CSN and/or baroreceptor region relative to the carotid artery.

In one aspect, the present disclosure provides a stimulation system including an implantable pulse generator (IPG) operably connected to an electrode lead with a cuff configured for circumferential or partial circumferential placement on or about a carotid artery, including but not limited to the internal carotid artery (ICA), common carotid artery (CCA), the external carotid artery (ECA), the carotid bulb, or the carotid sinus region. In preferred embodiments, the cuff is positioned on the ICA adjacent to the carotid sinus. The cuff is designed to encircle both the CSN and the carotid artery, thereby reducing or eliminating the need for surgical separation of the nerve from surrounding tissues. The cuff carries a plurality of electrically conductive contacts (or “electrodes”) on an inner surface to deliver stimulation to the CSN and/or to baroreceptors within the arterial wall. Any of the electrodes could be selectively used as an anode or a cathode to deliver stimulation or may be inactive. In various examples, the system is configured to induce a baroreflex response or otherwise modulate autonomic activity.

In some examples, the cuff body is formed from a flexible, biocompatible material, such as silicone, molded into a generally C-shaped (or horseshoe-shaped) body suitable for placement and retention on the exterior surface of a carotid artery. Following placement, the cuff body partially encircles the selected carotid artery segment through an arc of about 170° to about 270°. Although placement on or about the ICA is described in detail herein as a preferred embodiment due to proximity to the carotid sinus nerve, other carotid artery segments may be selected depending on anatomy and desired stimulation target. Compared with conventional cuffs that completely encircle the ICA, partial-circumferential or C-shaped cuffs of this disclosure have a smaller contact surface area, reducing the potential to cause irritation and avoiding arterial constriction while maintaining mechanical stability.

In some examples, lateral ends of the C-shaped cuff body terminate in outwardly extending suture anchor tabs (“tabs”) that function as fixation elements. Each tab (fixation element) includes at least one aperture for receiving a suture, allowing a surgeon to secure the cuff to muscle tissue in the vicinity of the ICA.

In some examples, the tabs incorporate compliance features, such as slots that extend through the tab, from a top or bottom edge, terminating inwardly from the opposing edge. Each slot may terminate at an arcuate or rounded transition to avoid stress risers in the comprising material. The slots increase the tab's flexibility, preventing or reducing the transmission of compressive forces from the cuff body to the ICA during muscular or arterial movements.

In another aspect, the stimulation cuff includes a cuff body with a concave inner surface that has a top edge, a bottom edge, a length direction defined between the top and bottom edges, opposing lateral edges, and a width direction defined between the lateral edges. The cuff body inner surface carries a plurality of electrodes arranged along a first direction and staggered along a second direction orthogonal to the first direction such that at least some electrodes overlap in the second direction. This arrangement substantially reduces or eliminates gaps between electrodes in the width direction and ensures that the CSN does not traverse between the electrodes. This arrangement accounts for interpatient variability in the CSN's location and course, ensuring that at least one electrode will lie across the CSN following cuff placement, even if the exact location of the CSN is visibly obscured by fibrous connective tissue. Accordingly, this configuration enables a surgeon to place the cuff in the safest and most mechanically advantageous position available without concern for electrode misalignment.

In some examples, the cuff body carries a plurality of electrodes arranged such that the electrodes are spaced along the length direction and staggered along a width direction, whereby each electrode at least partially overlaps another electrode in a plane extending in the length direction through the cuff body.

In some examples, each electrode completely overlaps one other electrode and partially overlaps one or more additional electrodes. Distribution of electrodes along the length of the cuff body (i.e., between the top and bottom edges) supports the formation of an electric field extending along a longitudinal axis of a carotid sinus nerve and/or across a baroreceptor region within a carotid artery, which may enhance physiological responses to stimulation by promoting the propagation of action potentials along the nerve length or activation of baroreceptors within the arterial wall.

In some examples, the cuff may include six total electrodes arranged in three pairs distributed along the first direction (corresponding to the length of the cuff body) and staggered along the second direction (corresponding to the width of the cuff body). In certain embodiments, each pair of electrodes comprises two completely overlapping electrodes, such that outer edges of the electrodes in a given pair are substantially colinear when viewed along the first direction.

In alternate embodiments, a cuff including one or more of the electrode arrangements described above could be configured to completely encircle ICA, with the cuff body extending 360° or more around ICA. In some cases, the cuff body is formed from a biocompatible, flexible polymeric material that can be moved between a furled and an unfurled state. In the unfurled state, the cuff body has a generally rectangular inner surface. The inner surface includes a stimulation region containing the electrodes and a compression region devoid of electrodes. The cuff body is materially biased to the furled state in an unstressed condition, can be manually unfurled for placement on the ICA, and returns to a furled state following placement. In the furled state, the compression region overlaps with the stimulation region, applying a slight compressive force that aids with retention without substantially constricting arterial pulsation. Following placement on the ICA, the cuff's lateral edges can remain unsecured, allowing the cuff body to expand to slightly unfurled states in response to arterial pulsations, thereby avoiding arterial constriction.

In some embodiments, the cuff body may be elastically conformable to accommodate variations in the diameter of a carotid artery. The cuff body may include compliant or resilient structures that allow the cuff to adjust to a range of carotid artery diameters while maintaining electrode contact with the arterial surface. In such embodiments, the cuff body may function as a self-sizing cuff by adapting to the artery without fixed coupling between opposing ends and without substantially restricting normal arterial pulsation, thereby maintaining stable electrode positioning despite anatomical variability.

In other implementations, the cuff body may include a stimulation region configured for placement on the carotid sinus, and one or more anchoring features, such as elongate fingers or extensions, which protrude downwardly and/or outwardly from the stimulation region and are secured circumferentially on a carotid artery segment to enhance fixation and stability of the implant.

In some examples, the IPG includes a receptacle to receive a lead connector and circuitry to generate stimulation, and may include a processor, memory, power source, clock, sensor(s), and a wireless communication unit for interaction with external devices such as a clinician programmer, a patient remote, and a charger. Additional components, features, and advantages will be explained in later sections of this disclosure.

### BRIEF DESCRIPTION OF THE DRAWINGS

Detailed descriptions of exemplary embodiments will be made with reference to the accompanying drawings.

FIG. 1 is an anatomical diagram of the human neck.

FIG. 2 is an enlarged view of a carotid artery segment, including the internal carotid artery, shown in FIG. 1.

FIG. 3 is a plan view of a stimulation system including an electrode lead secured to the internal carotid artery, in accordance with one embodiment.

FIG. 4 is a side view of a portion of the electrode lead illustrated in FIG. 3.

FIG. 5 is a block diagram of the stimulation system of FIG. 3 and optional external devices.

FIG. 6 is an isometric view of a first example embodiment for an electrode cuff configured for placement on the ICA.

FIG. 7 is a top, partial section of the ICA after placement of the cuff of FIG. 6.

FIG. 8 is a front view of the electrode cuff of FIG. 6, showing the arrangement of the electrodes.

FIG. 9 is an isometric view of a second example embodiment of an electrode cuff configured for placement on the ICA.

FIG. 10 is a top, partial section view of the cuff of FIG. 9 following placement on the ICA.

FIG. 11 is a front view of a third example embodiment of an electrode cuff, similar to the cuff of FIG. 10 but having structurally mirrored suture tabs.

FIG. 12 is another embodiment of an electrode cuff, shown in a furled state following placement on the ICA.

FIG. 13 is a front view of the cuff of FIG. 12 in a flattened, unfurled state.

FIG. 14 is the stimulation cuff of FIG. 13, shown in a furled state and viewed from the bottom.

### DETAILED DESCRIPTION

The following is a detailed description of the best presently known modes of carrying out the inventions. This description is not to be taken in a limiting sense but is made merely for the purpose of illustrating the general principles of the inventions. For example, although described in the context of tissue stimulators and electrode leads used in the treatment of high blood pressure, the present inventions are not so limited and are also applicable to implantable tissue stimulators and electrode leads configured to treat other medical conditions or to modulate autonomic activity associated with carotid sinus stimulation.

Unless stated otherwise, like-numbered elements refer to like parts throughout the drawings and description. Like-numbered elements need not necessarily be alike in all respects. Structural or functional differences could exist between like-numbered components associated with different examples.

As used herein, relative terms such as “top,” “bottom,” “left,” and “right” are used for convenience with respect to the orientation shown in the drawings and do not require a particular implantation orientation. In some instances, well-known structures and components are shown in block diagrams to avoid obscuring these concepts.

As used herein, the term “carotid artery” includes the common carotid artery (CCA), the internal carotid artery (ICA), the external carotid artery (ECA), the carotid bulb, and the carotid sinus region at or near the carotid bifurcation. Unless expressly stated otherwise, references to placement on the “internal carotid artery (ICA)” are intended to describe a preferred embodiment and are not limiting. The cuff body disclosed herein may be positioned on any suitable segment of the carotid artery where stimulation of the carotid sinus nerve (CSN) and/or baroreceptors may be achieved.

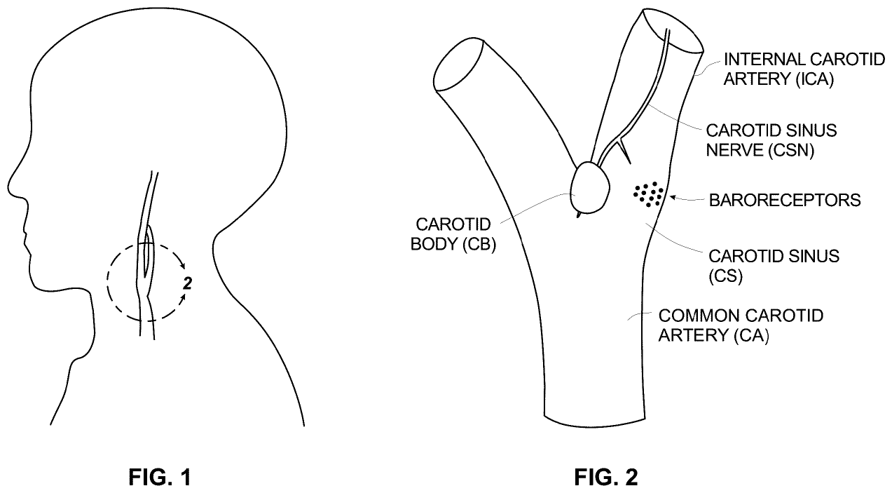

Briefly referring to the anatomical diagram shown in FIGS. 1-2, the present disclosure relates to a system and method for delivering stimulation to the Carotid Sinus Nerve (CSN) and/or to baroreceptors located within the carotid sinus region or arterial wall of a carotid artery. Stimulation may be applied at a location selected to directly activate the CSN, to activate baroreceptors embedded in the vascular wall, or to indirectly activate the CSN through vascular stimulation. The carotid sinus is located at the bifurcation of the common carotid artery and plays a crucial role in regulating blood pressure due to its involvement in the baroreceptor reflex (“baroreflex”) loop. For patients with hypertension, electrical stimulation of the CSN has been investigated as a treatment option to enhance, support, or replace its natural function in blood pressure regulation.

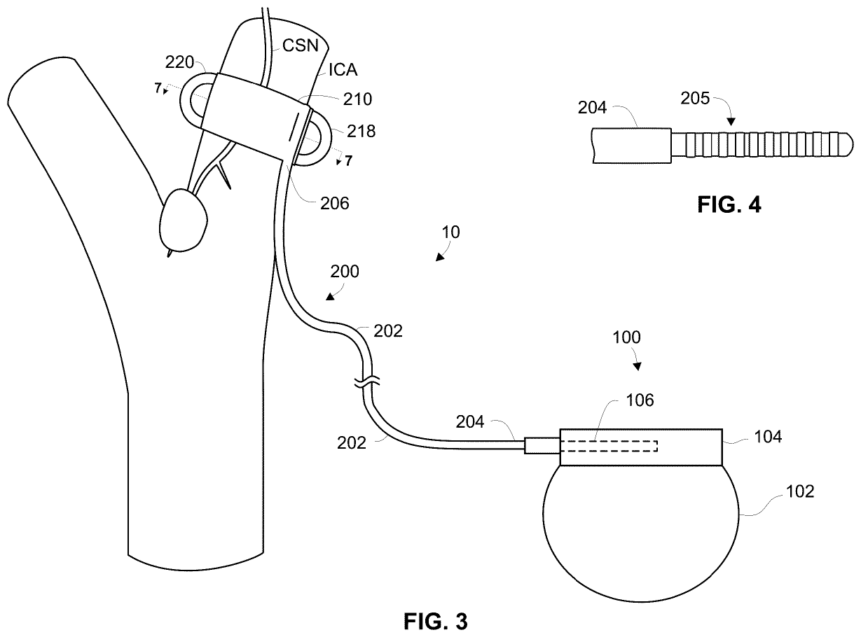

Referring to FIGS. 3-5, a stimulation system 10 is provided and includes an implantable stimulator, such as an implantable pulse generator 100 (“IPG” 100) and an electrode lead 200. The exemplary electrode lead 200 includes an arterial cuff (“cuff”) 210 adapted for circumferential placement on the ICA, and a lead body 202 that couples the cuff 210 to the IPG 100 by way of lead connector 205 with a plurality of electrical contacts on the proximal end 204 of lead body 202. The lead connector 205 is received by a receptacle 106 within a header on the IPG case 102, and elongated electrical conductors (e.g., wires) extend through the lead body to electrically couple contacts on the lead connector 205 to electrodes 28 carried by the cuff 210.

Optional external devices (collectively 300), such as a patient remote 300a, a charger 300b, and/or a clinician programmer 300c, may also be included in the system. External devices 300 could be configured to communicate wirelessly with IPG 100 using any known wireless communication modality having transmission and encryption protocols suitable for use in medical devices. For example, any of the external devices 300 may communicate with IPG 100 using Bluetooth, Bluetooth low energy, Wi-Fi, MedRadio, ultra-wideband, or the like.

The lead body 202 may include one or more S-shaped sections to provide strain relief, or it may be straight. The S-shaped sections accommodate body movement at the neck location where the lead body 202 is implanted, thereby reducing the likelihood of damage from unavoidable pulling of the electrode lead 200 that may result from neck movements. The accommodation provided by the S-shaped sections also reduces the likelihood of fatigue damage.

Although the exemplary system 10 shown in the drawings includes a single electrode lead 200, other embodiments may include a pair of electrode leads 200. In such cases, an additional electrode lead may be used to deliver baroreflex stimulation, to stimulate a different nerve (such as, e.g., the vagus nerve), or for sensing purposes. For instance, an additional lead (not shown) included within the system 10 could include one or more sensors for detecting a physiological signal or biomarker, such as heart rate, blood pressure, or neuronal activity. Such biomarkers can provide insight into patient health, disease state, and/or stimulation efficacy, and, in a closed-loop system, detection of a biomarker could be used as a trigger to start, stop, or otherwise modulate a stimulation parameter, such as pulse frequency, pulse width, pulse amplitude, or duty cycle.

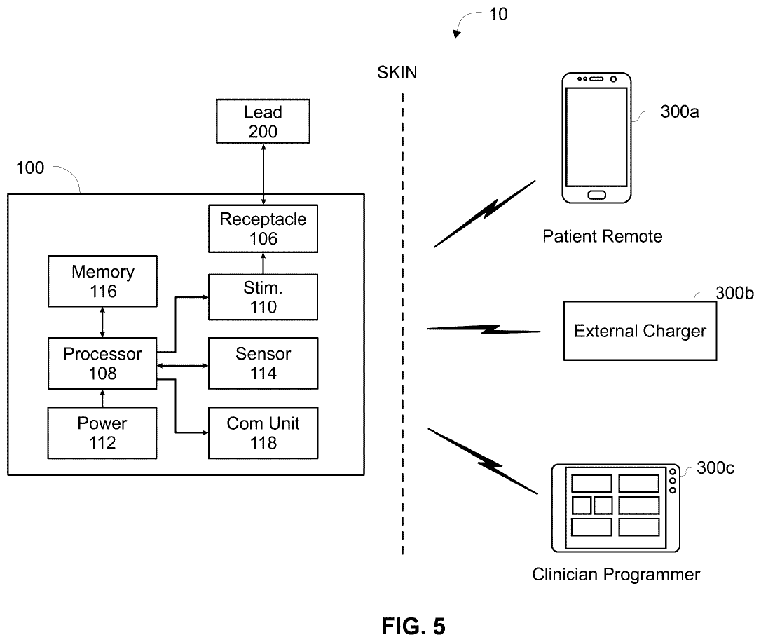

As schematically shown in FIG. 5, the stimulator 100 includes a processor 108, a stimulation circuit 110, a power source 112, one or more sensor(s) 114, memory 116, a communication unit 118, and a clock (e.g., RTC), each operably coupled. The receptacle 106 is in electrical communication with the stimulation circuit 110 to route signals to electrodes 28 on the cuff 210 via the lead 200. The communication unit 118 comprises a wireless transmitter, receiver, or transceiver to support secure wireless links with external device(s) 300.

In some examples, the one or more sensors 114 could comprise one or more of: an accelerometer, a gyroscope, a magnetometer, an inertial measurement unit (IMU), and/or an electrocardiogram (ECG). Such sensors may be used to monitor patient biomarkers, such as heart or respiratory rate, to track the patient's disease state or stimulation efficacy.

In some embodiments, the processor 108 can provide instructions to and receive information from the other components of the system 10. The processor can execute instructions stored in memory, associated with the processor 108, and/or in other components of the system 10. The processor 108 can, according to stored instructions, make decisions. In various examples, processor 108 might comprise a microprocessor, such as a microprocessor from Intel® or Advanced Micro Devices, Inc.®, or the like. The processor 108 can receive information and instructions from memory 116, and communicate with the stimulation circuit 110 to control when to deliver stimulation, which electrodes 28 are used, and for how long stimulation is delivered.

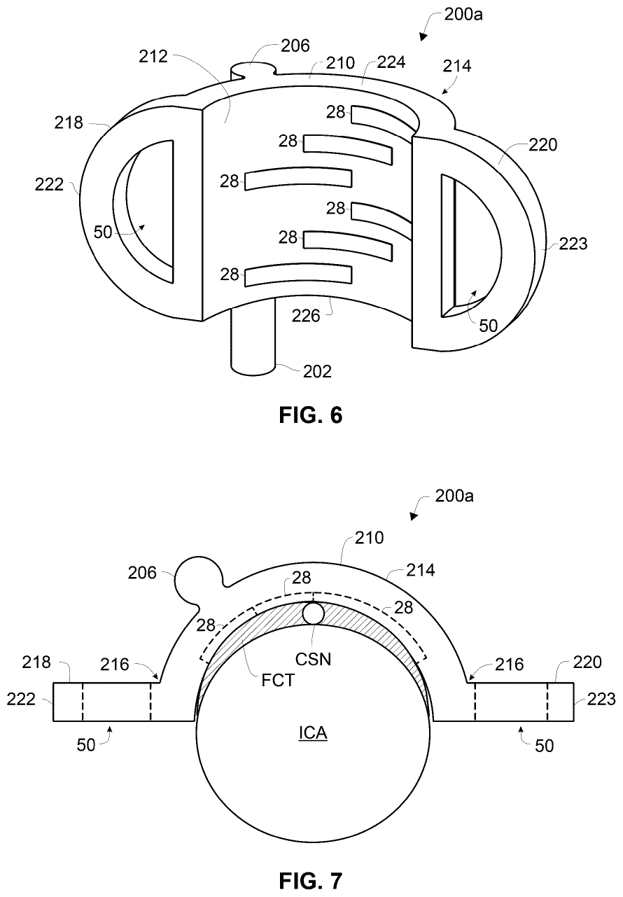

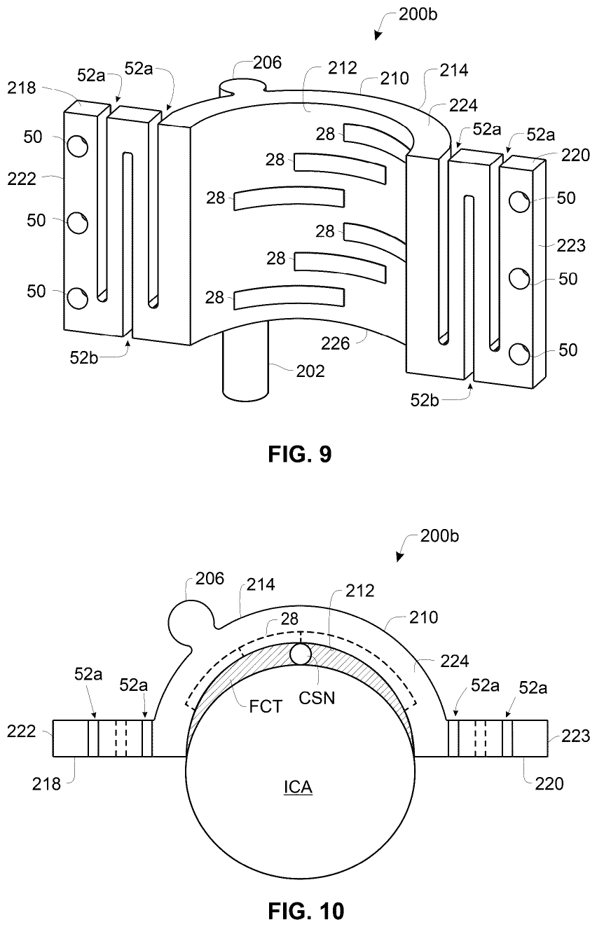

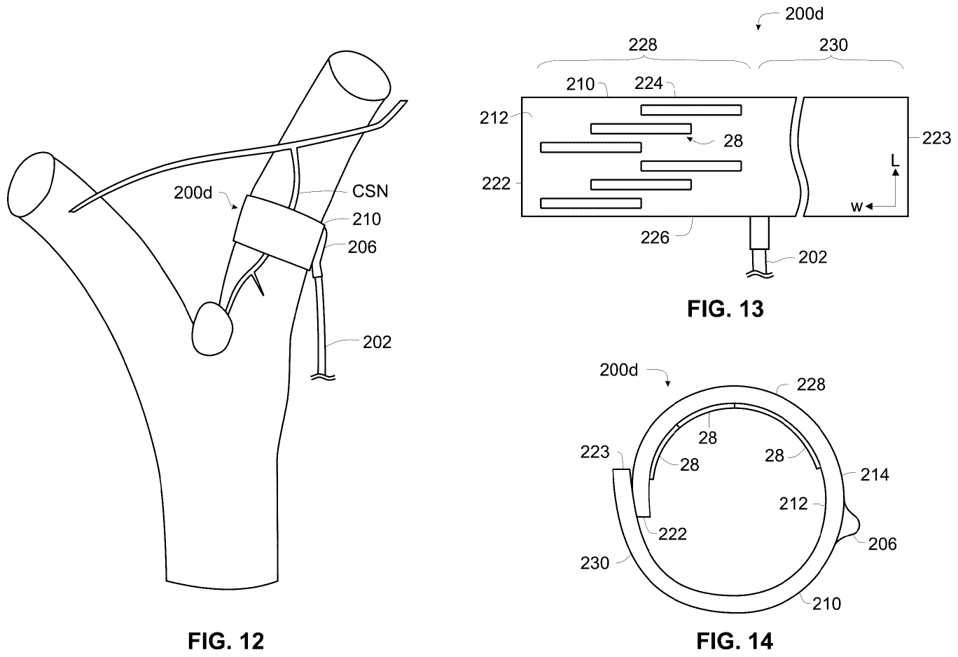

A first embodiment for an electrode lead 200a with a generally C-shaped cuff 210 will now be discussed in detail with reference to FIGS. 6-8. Other example embodiments for electrode leads 200b and 200c with generally C-shaped cuffs are discussed in later portions of this disclosure with reference to FIGS. 9-10 and FIG. 11, respectively, and a lead 200d including a circumferential cuff is discussed with reference to FIGS. 12-14. Although structural features may vary between the various cuff embodiments 200a-200d, some features described in relation to the first C-shaped cuff embodiment 200a, particularly those that pertain to the particular configuration or spacing of electrodes 28 on the cuff body inner surface 212, apply to each of the example leads 200a-200d and may therefore be excluded from explicit discussion in the later embodiments.

Referring to FIGS. 3-4 and 6-8, lead 200a includes a lead body 202 having a proximal end 204 terminating in the connector 205 and a distal end 206 coupled to a cuff body 210. The cuff body 210 is formed of a flexible, biocompatible, and electrically insulative material (e.g., silicone) having an inner surface 212 configured to abut the ICA, a plurality of electrically conductive contacts 28 (or “electrodes” 28) carried by the inner surface 212, and an outer surface 214 disposed opposite the inner surface 212. Suitable materials for the electrodes 28 include, but are not limited to, platinum-iridium and palladium.

The cuff body 210 is generally C-shaped, with a concave inner surface 212 and a convex outer surface 214. Depending on the implementation, inner surface 212 may have an internal radius of about 2.5 mm to 3.5 mm, sized to conform to the exterior surface of the ICA, which typically has an exterior diameter of about 6 mm to 7 mm. The cuff body inner surface 212 extends in a partial arc around the ICA, with opposing sides including transition regions 216 from which fixation elements, such as suture tabs 218 and 220, extend outwardly from cuff body 210. The suture tabs 218 and 220 terminate at free ends 222 and 223, respectively

In the sectional diagram shown in FIGS. 7 and 10, the cuff body is shown from a top perspective following placement on the ICA. As shown, the cuff body extends around the ICA in an arc roughly 170 degrees about the artery's circumference, although different implementations may extend between 170 and 270 degrees. As shown, the CSN is often encased in a sheath of fibrous connective tissue (FCT), making it difficult for a surgeon to visually discern the CSN's exact location and course. However, the CSN generally lies on the anterior wall of the ICA, and the staggered electrode distribution accommodates variable CSN positioning, ensuring that at least one electrode 28 lies across the CSN following cuff placement. The illustrated placement on the internal carotid artery is exemplary and non-limiting. In other embodiments, the cuff may be positioned on the common carotid artery, the carotid bulb, or another suitable segment proximate to the carotid sinus.

As noted, at the intended implantation location (e.g., FIG. 3), the carotid artery has a much larger (6-7 mm) diameter than the CSN (0.5-1.5 mm). In addition, the artery walls are more mechanically robust than the epineurium of the CSN. Wrapping the cuff body 210 around the artery rather than the nerve provides the device with a strong mechanical base and mitigates any pressure or other forces the cuff may impart on the nerve, while also avoiding the need to surgically separate the CSN from the ICA. When positioned over the carotid sinus region, stimulation may additionally activate baroreceptors within the arterial wall.

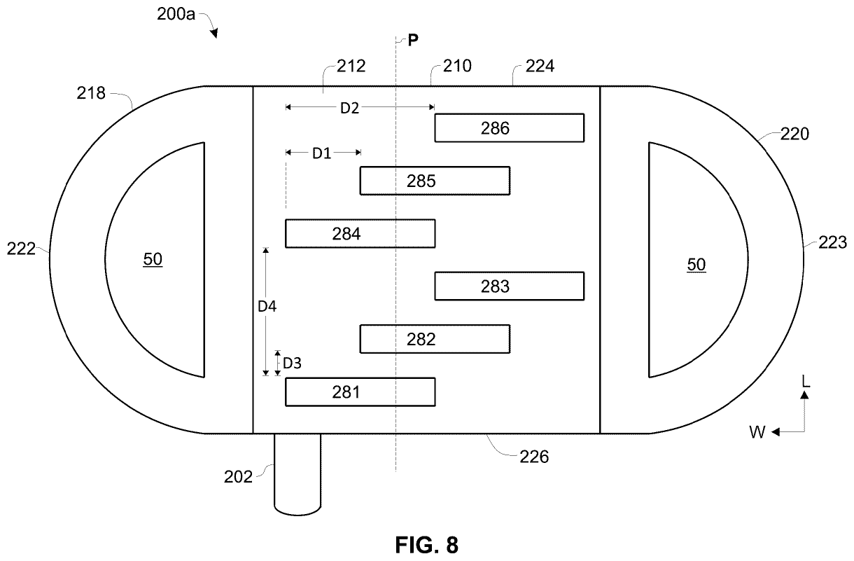

Referring to FIG. 8, the cuff body 210 carries or includes a plurality of electrodes (collectively 28), which in this example includes six electrodes 281-286 on the inner surface 212. Electrodes 281-286 are arranged (i.e., spaced apart) along a first direction (L) of the cuff body 210. The electrodes 281-286 are staggered along a second direction (W) orthogonal to the first direction such that each electrode (any of 28) at least partially overlaps another electrode in the first direction. In the illustrated embodiment, electrodes 281 and 284 form a first overlapping pair, electrodes 282 and 285 form a second overlapping pair, and electrodes 283 and 286 form a third overlapping pair, with each electrode of a pair positioned along the first direction and laterally offset relative to adjacent electrodes along the second direction. In some examples, at least one electrode completely overlaps another electrode in a plane (P) extended along the first direction of the cuff body. As shown, each electrode (any one of 28) completely overlaps one other electrode (any other of 28) and partially overlaps one or more additional electrodes (any one or more other of 28).

By way of example, electrode 281 is toward the left side and nearest the bottom edge 226 and completely overlaps electrode 284; electrode 282 is centrally located, completely overlapping with electrode 285 in the first direction and partially overlapping electrodes 281, 283, 284, and 286; electrode 283 is toward the right side and completely overlaps with electrode 286. In the illustrated embodiment, adjacent electrodes are laterally offset relative to one another along the second direction (W), forming three overlapping electrode pairs distributed along the first direction (L).

Depending on the implementation, any two or more of the electrodes (281-286) included on the cuff body 210 may be selectively activated as anodes or cathodes for delivering bipolar stimulation.

In a monopolar stimulation mode, any one of the electrodes (any one of 281-286) could be activated as a cathode, forming an anode-cathode pair with an electrically conductive portion of the IPG case 102, which functions as an indifferent anode or return electrode.

In some embodiments, electrodes that overlap in the first direction (L) may be activated together or independently to shape an electric field extending along the first direction (L) of cuff body 210. The staggered arrangement shown in FIG. 8 permits selection of different electrode combinations to account for anatomical variability in the position of a carotid sinus nerve or baroreceptors relative to the cuff body.

Referring to FIG. 8, adjacent electrodes arranged along the first direction (L) are separated from one another by spacing distances that may be selected based on desired stimulation characteristics. The distribution of the electrodes along the first direction supports the formation of an electrical field extending generally along the first direction of the cuff body 210 and along a longitudinal axis of a carotid sinus nerve when positioned adjacent the cuff body, which may improve the strength of an induced baroreflex response by facilitating signal propagation along the nerve length. The staggered arrangement of the electrodes along the second direction (W) improves coverage across the width of the cuff body and enhances the likelihood that at least one electrode will overlie a carotid sinus nerve or baroreceptor region despite anatomical variability.

As shown, each successive electrode (e.g., 281 to 282, or 284 to 285) is spaced apart in the first direction (L) by a distance D3, and offset in the second direction (W) by distances D1 or D2. Completely overlapping electrode pairs (e.g., 281 and 284) may be separated along the first direction by a selected spacing distance D4 that is greater than D3. The specific spacing and offset distances may vary depending on electrode size, desired electric field distribution, and anatomical considerations.

In some embodiments, two or more electrodes distributed along the first direction of the cuff body may be selectively activated to generate an electrical field extending generally along the first direction. Activation of electrodes positioned at different locations along the first direction may produce an electric field component aligned with the longitudinal axis of a carotid sinus nerve when the cuff body is positioned on a carotid artery. The particular electrodes selected for activation may be varied to shape, direct, or adjust the magnitude of the electrical field extending along the first direction to stimulate a carotid sinus nerve and/or baroreceptors.

In some embodiments, the electrodes disposed on the cuff body may have various geometries and configurations, including rectangular, circular, elongated, or other shapes. The size, shape, spacing, and overlap of the electrodes may be selected to control current density, field distribution, and selectivity of neural or baroreceptor activation, and may be combined with the staggered arrangements described above or used in alternative spatial configurations.

Suture tabs 218, 220 extend outwardly from the cuff body 210 at transition regions 216 (best shown in FIG. 7) and include apertures 50 configured to receive sutures for securing the cuff body to adjacent tissue in proximity to a carotid artery. The tabs may be positioned at opposing lateral regions of the cuff body or at any suitable location to promote stable fixation.

Referring to FIGS. 6-8, tabs 218 and 220 may comprise loop-shaped structures, each including at least one aperture 50. The tabs 218 and 220 may be integrally formed with the cuff body 210 or formed separately and attached thereto. In some embodiments, the tabs may have a modulus of elasticity that differs from that of the cuff body to permit enhanced flexibility or strain relief during neck movement or arterial pulsation.

Referring to FIGS. 9-10, a second embodiment for a lead 200b includes a generally C-shaped cuff body 210 having tabs 218 and 220. Each tab 218 and 220 is generally rectangular and includes a plurality of slots (collectively 52), including upper slots 52a and lower slots 52b, arranged in an alternating pattern. The slots 52 allow the tabs 218 and 220 to deform resiliently in response to arterial or muscular movement. The upper slots 52a extend from the top edge 224, and the lower slots 52b extend from the bottom edge 226. The slots 52a, 52b terminate short of the opposing edge and may include arcuate (or rounded) terminal ends for the purpose of reducing stress risers in the comprising material. Such slots are one example of a compliance feature that permit the tab to flex while the cuff body 210 remains positioned on the carotid artery. Alternate embodiments for such tabs may include one or more slots 52 that extend vertically through the tab (as shown), at an angle through the tab (218 or 220), or curvilinearly through the tab (218 or 220).

The tabs 218 and 220 of cuff 210 on lead 200b each include three apertures 50 near respective outer edges 222 and 223. The apertures 50 are distributed vertically (i.e., spaced along the length direction) to provide a surgeon with a variety of suture placement options, enabling the surgeon to choose any one or more apertures on each tab 218, 220 to suture the tab to a convenient location.

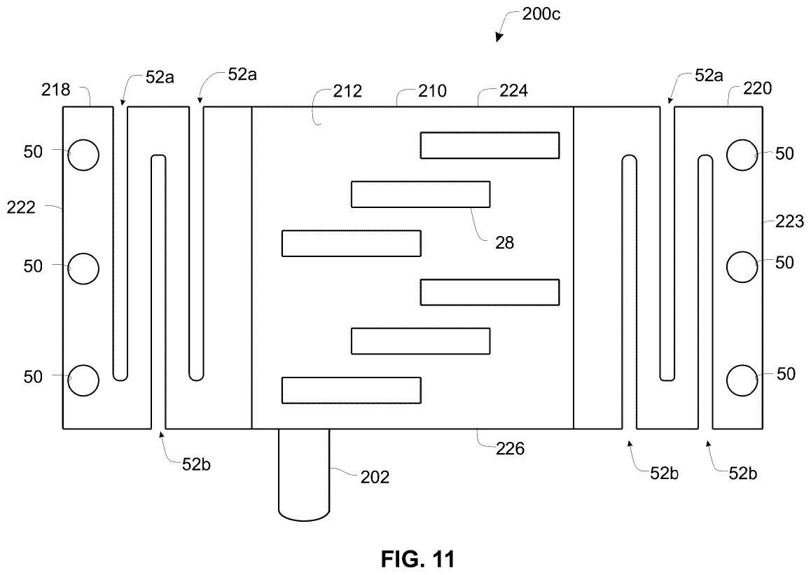

Referring to FIG. 11, a third embodiment for a lead 200c is provided including tabs 218 and 220 with compliant features similar to those of lead 200b (shown in FIG. 9). However, the slot pattern of lead 200c in FIG. 11 is mirrored across a horizontal plane, such that the right tab 220 includes two lower slots 52b separated by one upper slot 52a, while the left tab 218 includes two upper slots 52a separated by one lower slot 52b. This structural mirroring improves the balance of the tabs following fixation to nearby tissues (via sutures through apertures 50) and prevents the cuff body 210 from bowing under tension if the tabs are fully extended.

Referring to FIGS. 12-14, a fourth embodiment for an electrode lead (“lead”) 200d is shown. Lead 200d includes six electrodes 28 with a configuration like those of leads 200a-200c but includes a cuff body 210 configured to wrap more than 360° around the ICA. In an unstressed state, the cuff body 210 is materially biased to a furled position, can be manually unfurled for circumferential placement on the ICA, and refurled to retain itself upon the ICA without securing free ends 222 and 223 of the cuff body 210 to one another. The cuff includes a stimulation region 228 containing electrodes 28 and a compression region 230 devoid of electrodes 28.

In FIG. 14, cuff body 210 of lead 200d is shown from a side view in a furled or partially furled state, showing how the compression region 230 overlaps at least a portion of stimulation region 228. The overlap may apply a retention force sufficient to maintain the cuff body on the carotid artery while permitting expansion in response to arterial pulsation.

Although the inventions disclosed herein have been described in terms of the preferred embodiments above, numerous modifications and/or additions to the above-described preferred embodiments would be readily apparent to one skilled in the art. It is intended that the scope of the present inventions extend to all such modifications and/or additions. The inventions include any and all combinations of the elements from the various embodiments disclosed in the specification. The scope of the present inventions is limited solely by the claims set forth below.

What is claimed is:

1. An implantable electrode lead, comprising:
an elongate lead body; and
a cuff body coupled to the elongate lead body and configured for placement on or about a carotid artery;
wherein the cuff body includes a plurality of electrodes spaced apart along a first direction of the cuff body and staggered along a second direction orthogonal to the first direction, such that at least some of the electrodes partially overlap in the first direction.

2. The electrode lead of claim 1, wherein the carotid artery comprises the internal carotid artery.

3. The electrode lead of claim 1, wherein at least one electrode completely overlaps another electrode in the first direction.

4. The electrode lead of claim 3, wherein the at least one electrode partially overlaps two or more electrodes in the first direction.

5. The electrode lead of claim 1, wherein the plurality of electrodes comprises six electrodes.

6. The electrode lead of claim 1, wherein the plurality of electrodes are positioned inwardly from the top edge and the bottom edge of the cuff body.

7. The electrode lead of claim 1, wherein the electrode arrangement is configured to maximize the likelihood that at least two electrodes are placed across a carotid sinus nerve.

8. The electrode lead of claim 1, wherein each of the plurality of electrodes is selectively configurable as an anode or a cathode for delivering bipolar or monopolar stimulation.

9. The electrode lead of claim 8, wherein, in a bipolar stimulation mode, the distribution of the electrodes along the first direction is configured to facilitate formation of an electrical field that extends along the first direction of the cuff body and along a longitudinal axis of a carotid sinus nerve.

10. The electrode lead of claim 1, wherein the cuff body is configured to wrap circumferentially more than 360 degrees around a carotid artery.

11. The electrode lead of claim 10, wherein the cuff body includes first and second ends that remain unsecured to one another when the cuff body is placed on the carotid artery.

12. The electrode lead of claim 11, wherein the cuff body is materially biased toward a furled state to retain the cuff body on the carotid artery.

13. The electrode lead of claim 1, wherein the lead body includes a proximal connector configured for insertion into a receptacle of an implantable pulse generator.

14. The electrode lead of claim 11, wherein the lead body includes elongate conductors electrically coupling the proximal connector to the plurality of electrodes.

15. An implantable electrode lead, comprising:
a lead body;
a cuff body coupled to the cuff body and configured for placement on or about a carotid artery, the cuff body carrying at least one electrode,
wherein the cuff body is configured to extend circumferentially about a carotid artery through an arc between about 170 degrees and about 270 degrees.

16. The implantable electrode lead of claim 15, including at least one fixation element extending from the cuff body.

17. The implantable electrode lead of claim 16, wherein the at least one fixation element includes an aperture configured to receive a suture to secure the cuff body to tissue adjacent the carotid artery.

18. The implantable electrode lead of claim 17, wherein the fixation element includes a compliance feature configured to accommodate movement of the carotid artery while the cuff body remains positioned on the carotid artery.

19. The electrode lead of claim 18, wherein the compliance feature comprises one or more slots formed in the fixation element.

20. The electrode lead of claim 19, wherein the one or more slots extend inwardly from a peripheral edge of the fixation element and terminate prior to reaching an opposing peripheral edge.

21. The electrode lead of claim 20, wherein each slot terminates in an arcuate end portion.

22. The electrode lead of claim 19, wherein the compliance feature allows resilient extension of the fixation element while the cuff body remains seated on the carotid artery.

23. The electrode lead of claim 22, wherein the compliance feature is configured to accommodate movement associated with head or neck motion.

24. The electrode lead of claim 15, wherein the at least one electrode comprises a plurality of electrodes arranged on a surface of the cuff body configured to face the carotid artery.

25. The electrode lead of claim 19, wherein the plurality of electrodes are arranged along a first direction of the cuff body and staggered along a second direction orthogonal to the first direction such that at least some of the electrodes overlap in the first direction.
